(19) United States
(12) Patent Application Publication
(10) Pub. No.: US 2026/0294647 A1
(43) Pub. Date: Oct. 1, 2026

(54) LINKED RESECTION INSTRUMENT AND METHOD OF USING THE SAME IN AN ORTHOPAEDIC SURGICAL KNEE PROCEDURE

(71) Applicant: DePuy Ireland Unlimited Company, Ringaskiddy (IE)

(72) Inventors: Jason M. Chavarria, Warsaw, IN (US); Jeremiah M. Lewis, Warsaw, IN (US)

(21) Appl. No.: 19/417,910

(22) Filed: Dec. 12, 2025

Related U.S. Application Data
(60) Provisional application No. 63/780,734, filed on Mar. 31, 2025.

(51) Int. Cl. A61F 2/46; A61B 17/15; A61B 17/17; A61B 17/56

(52) U.S. Cl. CPC A61F 2/461; A61B 17/155; A61B 17/157; A61B 17/1764; A61B 2017/564

(57) ABSTRACT

A linked resection instrument includes an elongated body, a femoral pin guide, and a tibial pin guide. The distance between the femoral and tibial pin guides is adjustable based on the thickness of a knee prosthesis to be implanted. The anteroposterior angle of the tibial pin guide is adjustable based on a desired posterior slope angle of the knee prosthesis to be implanted. Methods of surgically preparing a patient’s femur and tibia is also disclosed.

This application claims priority to U.S. Provisional Patent Application Serial No. 63/780,734 which was filed on Mar. 31, 2025, which is hereby incorporated by reference in its entirety.

### TECHNICAL FIELD

The present disclosure relates generally to orthopaedic surgical instruments and, more particularly, to surgical instruments used during an orthopaedic surgical knee procedure.

### BACKGROUND

Joint arthroplasty is a well-known surgical procedure by which a diseased and/or damaged natural joint is replaced by a prosthetic joint. For example, in a total knee arthroplasty surgical procedure, a patient’s natural knee joint is partially or totally replaced by a prosthetic knee joint or knee prosthesis. To facilitate the replacement of the natural joint with the prosthesis, orthopaedic surgeons use a variety of orthopaedic surgical instruments such as, for example, saws, drills, reamers, rasps, broaches, cutting blocks, drill guides, milling guides, and other surgical instruments.

In total knee arthroplasty (TKA), the femur and tibia of the patient’s knee are resected to create planar surfaces onto which a prosthetic femoral component and tibial component, respectively, are installed. Traditional TKA involves determining the resection planes based on a pre-determined angle as a function of mechanical alignment or by using a balanced approach that sets the resection planes based on ligament tension. More recently, kinematic alignment techniques involve determining the resection planes as a function of the native, pre-disease state of the patient’s knee.

### SUMMARY

According to one aspect of the disclosure, an orthopaedic surgical instrument assembly for use during an orthopaedic knee replacement procedure includes an elongated body, a femoral pin guide, a tibial pin guide, and a slope adjustment collet. The femoral pin guide is positioned on the elongated body and is slidable along the elongated body in the superoinferior direction. The tibial pin guide is pivotally coupled to the elongated body at a location inferior to the femoral pin guide. The slope adjustment collet is rotatably coupled to the elongated body and has a tapered outer surface. Rotation of the slope adjustment collet in a first direction urges the collet’s tapered outer surface toward the tibial pin guide so as to increase an anteroposterior pin angle of the tibial pin guide. Rotation of the slope adjustment collet in a second, opposite direction urges the collet’s tapered outer surface away from the tibial pin guide so as to decrease the anteroposterior pin angle of the tibial pin guide.

In an embodiment, the femoral pin guide includes a pair of pin guide holes configured to locate placement of a distal femoral cutting block on an anterior surface of a femur of a patient.

In an embodiment, the tibial pin guide includes a flange having a pair of pin guide holes configured to locate placement of a tibial cutting block on an anterior surface of a tibia of a patient. The elongated body has a longitudinal axis that defines the anteroposterior pin angle with a longitudinal axis of the pair of pin guide holes. The flange is pivotally coupled to the elongated body via a pivot joint such that rotation of the flange relative to the elongated body at the pivot joint selectively alters the anteroposterior pin angle.

In an embodiment, the tibial pin guide has a cam surface formed therein. Rotation of the slope adjustment collet in the first direction moves the collet’s tapered outer surface inferiorly along the cam surface of the tibial pin guide so as to increase the anteroposterior pin angle of the tibial pin guide, whereas rotation of the slope adjustment collet in the second, opposite direction urges the collet’s tapered outer surface superiorly along the cam surface of the tibial pin guide so as to decrease the anteroposterior pin angle of the tibial pin guide.

In an embodiment, the elongated body has a superior body segment having a smooth outer surface and a middle body segment having a threaded outer surface. The femoral pin guide has an elongated bore that is positioned on the superior body segment of the elongated body so as to allow the femoral pin guide to superinferiorly translate along the smooth outer surface. The slope adjustment collet has a threaded bore that is threadingly-engaged with the threaded outer surface so as to allow the slope adjustment collet to superinferiorly translate along the elongated body’s middle body segment.

In an embodiment, the orthopaedic surgical instrument assembly includes a locking knob operable to lock the position of the femoral pin guide relative to the elongated body.

In an embodiment, the orthopaedic surgical instrument assembly also includes a femoral alignment rod secured to a superior end of the elongated body and extending superiorly away therefrom, and a tibial alignment rod secured to an inferior end of the elongated body and extending inferiorly away therefrom.

According to another aspect, a method of surgically preparing a patient’s femur and tibia during an orthopaedic surgical knee procedure includes installing a pair of femoral alignment pins in an anterior surface of the patient’s femur, and thereafter installing a femoral pin guide of a linked resection instrument onto the pair of installed femoral alignment pins. A distance between the femoral pin guide of the linked resection instrument and a tibial pin guide of the linked resection instrument is adjusted based on a thickness of a knee prosthesis to be implanted. An anteroposterior pin angle of the tibial pin guide of the linked resection instrument is adjusted based on a desired posterior slope angle of the knee prosthesis to be implanted. A pair of tibial alignment pins is installed in an anterior surface of the patient’s tibia with the tibial pin guide of the linked resection instrument.

A femoral alignment jig may be operated to install the pair of femoral alignment pins in the anterior surface of the patient’s femur.

In an embodiment, the method also includes aligning a tibial alignment rod of the linked resection instrument with an ankle of the patient, and aligning a femoral alignment rod of the linked resection instrument with a hip of the patient.

In an embodiment, the method includes assessing changes to the distance between the femoral pin guide of the linked resection instrument and the tibial pin guide of the linked resection instrument as a result of adjustment of the anteroposterior pin angle, and then readjusting the distance between the femoral pin guide of the linked resection instrument and the tibial pin guide of the linked resection instrument.

In an embodiment, the method also includes installing a distal femoral cutting block on the installed femoral alignment pins, and thereafter resecting a distal end of the patient’s femur with the installed distal femoral cutting block.

In an embodiment, the method also includes installing a tibial cutting block on the installed tibial alignment pins, and thereafter resecting a proximal end of the patient’s tibia with the installed tibial cutting block.

According to another aspect, a method of surgically preparing a patient’s femur and tibia during an orthopaedic surgical knee procedure includes installing a pair of tibial alignment pins in an anterior surface of the patient’s tibia, and installing a tibial pin guide of a linked resection instrument onto the pair of installed tibial alignment pins. A distance between the tibial pin guide of the linked resection instrument and a femoral pin guide of the linked resection instrument is adjusted based on a thickness of a knee prosthesis to be implanted. An anteroposterior pin angle of the tibial pin guide of the linked resection instrument is adjusted based on a desired posterior slope angle of the knee prosthesis to be implanted. A pair of femoral alignment pins is installed in an anterior surface of the patient’s femur with the femoral pin guide of the linked resection instrument.

A tibial alignment jig may be operated to install the pair of tibial alignment pins in the anterior surface of the patient’s tibia.

In an embodiment, the method also includes aligning a tibial alignment rod of the linked resection instrument with an ankle of the patient, and aligning a femoral alignment rod of the linked resection instrument with a hip of the patient.

In an embodiment, the method includes assessing changes to the distance between the femoral pin guide of the linked resection instrument and the tibial pin guide of the linked resection instrument as a result of adjustment of the anteroposterior pin angle, and then readjusting the distance between the femoral pin guide of the linked resection instrument and the tibial pin guide of the linked resection instrument.

In an embodiment, the method also includes installing a distal femoral cutting block on the installed femoral alignment pins, and thereafter resecting a distal end of the patient’s femur with the installed distal femoral cutting block.

In an embodiment, the method also includes installing a tibial cutting block on the installed tibial alignment pins, and thereafter resecting a proximal end of the patient’s tibia with the installed tibial cutting block.

In an embodiment, the method also includes installing a ligament balancer between the patient’s tibia and femur before installing the pair of femoral alignment pins in the anterior surface of the patient’s femur with the femoral pin guide of the linked resection instrument.

According to another aspect, a method of surgically preparing a patient’s femur and tibia during an orthopaedic surgical knee procedure includes installing a pair of tibial alignment pins in an anterior surface of the patient’s tibia, and installing a tibial pin guide of a linked resection instrument onto the pair of installed tibial alignment pins. A distance between the tibial pin guide of the linked resection instrument and a femoral pin guide of the linked resection instrument is adjusted based on a thickness of a knee prosthesis to be implanted. An anteroposterior pin angle of the tibial pin guide of the linked resection instrument is adjusted based on a desired posterior slope angle of the knee prosthesis to be implanted. The method also includes installing a ligament balancer between the patient’s tibia and femur. With the ligament balancer installed between the patient’s tibia and femur, a pair of femoral alignment pins is installed in an anterior surface of the patient’s femur with the femoral pin guide of the linked resection instrument.

A tibial alignment jig may be operated to install the pair of tibial alignment pins in the anterior surface of the patient’s tibia.

In an embodiment, the method also includes aligning a tibial alignment rod of the linked resection instrument with an ankle of the patient, and aligning a femoral alignment rod of the linked resection instrument with a hip of the patient.

In an embodiment, the method includes assessing changes to the distance between the femoral pin guide of the linked resection instrument and the tibial pin guide of the linked resection instrument as a result of adjustment of the anteroposterior pin angle, and then readjusting the distance between the femoral pin guide of the linked resection instrument and the tibial pin guide of the linked resection instrument.

In an embodiment, the method also includes installing a distal femoral cutting block on the installed femoral alignment pins, and thereafter resecting a distal end of the patient’s femur with the installed distal femoral cutting block.

In an embodiment, the method also includes installing a tibial cutting block on the installed tibial alignment pins, and thereafter resecting a proximal end of the patient’s tibia with the installed tibial cutting block.

With the patient’s femur and tibia positioned in extension, the ligament balancer may be installed between an unresected proximal end of the patient’s tibia and an unresected distal end of the patient’s femur.

According to yet another aspect, a method of surgically preparing a patient’s femur and tibia during an orthopaedic surgical knee procedure includes adjusting a distance between a tibial pin guide of a linked resection instrument and a femoral pin guide of the linked resection instrument based on a thickness of a knee prosthesis to be implanted, and adjusting an anteroposterior pin angle of the tibial pin guide of the linked resection instrument based on a desired posterior slope angle of the knee prosthesis to be implanted. The method also includes aligning a tibial alignment rod of the linked resection instrument with an ankle of the patient, and aligning a femoral alignment rod of the linked resection instrument with a hip of the patient. The method further includes installing a pair of tibial alignment pins in an anterior surface of the patient’s tibia with the tibial pin guide of the linked resection instrument, and installing a pair of femoral alignment pins in an anterior surface of the patient’s femur with the femoral pin guide of the linked resection instrument.

In an embodiment, the method includes assessing changes to the distance between the femoral pin guide of the linked resection instrument and the tibial pin guide of the linked resection instrument as a result of adjustment of the anteroposterior pin angle, and then readjusting the distance between the femoral pin guide of the linked resection instrument and the tibial pin guide of the linked resection instrument.

In an embodiment, the method also includes installing a distal femoral cutting block on the installed femoral alignment pins, and thereafter resecting a distal end of the patient’s femur with the installed distal femoral cutting block.

In an embodiment, the method also includes installing a tibial cutting block on the installed tibial alignment pins, and thereafter resecting a proximal end of the patient’s tibia with the installed tibial cutting block.

### BRIEF DESCRIPTION OF THE DRAWINGS

The detailed description particularly refers to the following figures, in which:

FIG. 1 is a fragmentary front view of a linked resection instrument for use in the surgical preparation of a patient’s femur and tibia during performance of an orthopaedic knee procedure;

FIG. 2 is a fragmentary side view of the linked resection instrument of FIG. 1;

FIG. 3 is a cross sectional view of the linked resection instrument taken along the line 3-3 of FIG. 1, as viewed in the direction of the arrows;

FIG. 4 is a view similar to FIG. 3, but showing slope adjustment collet having been used to increase the posterior slope angle of the linked resection instrument;

FIG. 5 is a front view of the linked resection instrument of FIG. 1 installed in the patient’s knee;

FIG. 6 is an enlarged fragmentary front view of the linked resection instrument of FIG. 1 installed in the patient’s knee;

FIG. 7 is an enlarged fragmentary side view of the linked resection instrument of FIG. 1 installed in the patient’s knee; and

FIG. 8 is an enlarged front view of a distal femoral cutting block and a tibial cutting block installed in the patient’s knee.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, proximal, distal, etcetera, may be used throughout the specification in reference to the orthopaedic implants and surgical instruments described herein as well as in reference to the patient’s natural anatomy. Such terms have well-understood meanings in both the study of anatomy and the field of orthopaedics. Use of such anatomical reference terms in the written description and claims is intended to be consistent with their well-understood meanings unless noted otherwise.

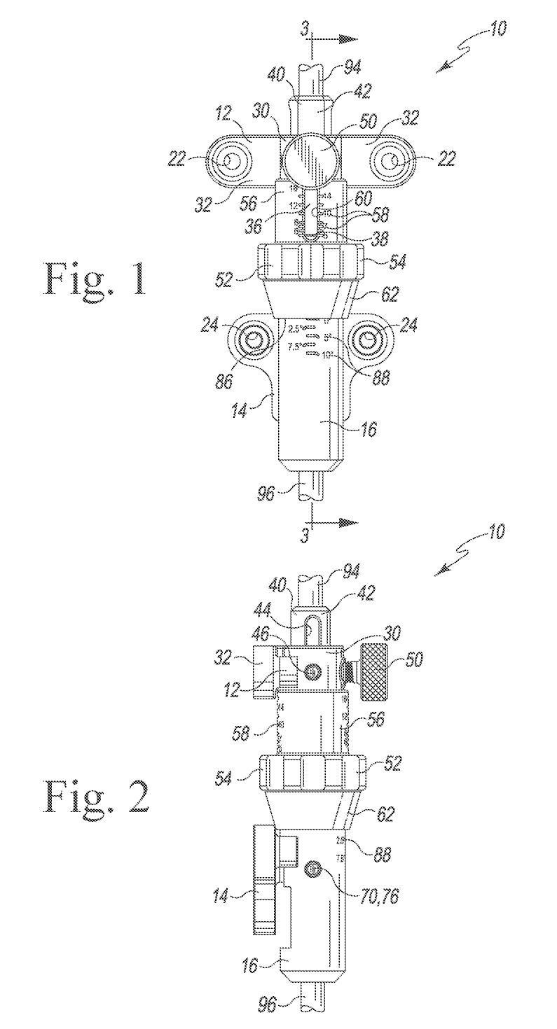

Referring to FIGS. 1-4, an orthopaedic surgical instrument 10 - in the form of a linked resection instrument - for use in the surgical preparation of a patient’s femur and tibia during performance of an orthopaedic knee procedure is shown. As will be described below in more detail, the linked resection instrument 10 may be used by a surgeon to position a distal femoral cutting block on the distal end of the patient’s femur and a tibial cutting block on the proximal end of the patient’s tibia. Because both cutting blocks are placed with a common instrument, their locations and orientations are linked to one another.

The linked resection instrument 10 may be used both by surgeons with a preference for mechanical alignment techniques and those surgeons with a preference for kinematic alignment techniques. Broadly, mechanical alignment techniques involve determining the resection planes based on a pre-determined angle as a function of mechanical alignment, whereas kinematic alignment techniques involve determining the resection planes as a function of the native, pre-disease state of the patient’s knee. Moreover, as will be described in more detail below, the linked resection instrument 10 may also be used in an inverse kinematic alignment technique in which ligament balancing is used in conjunction with a kinematic alignment technique.

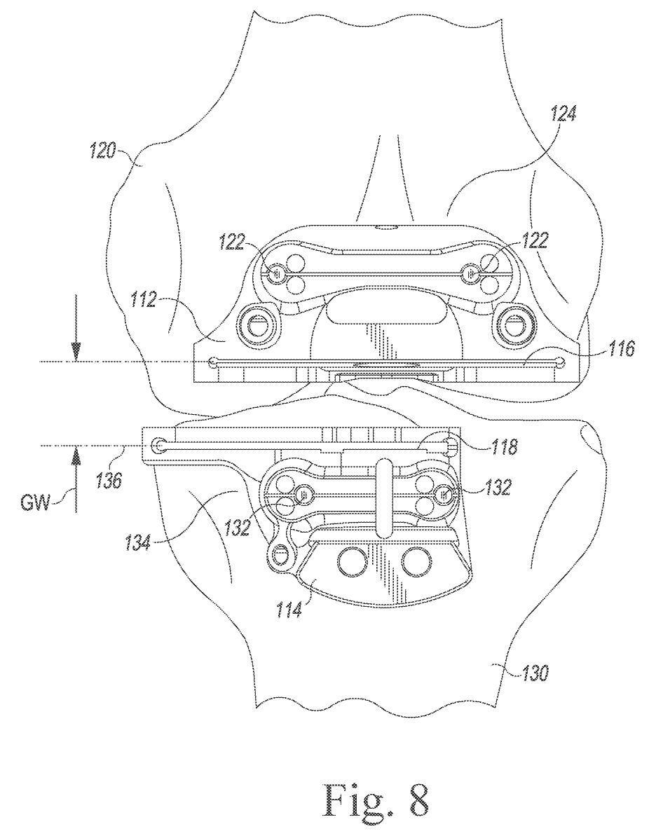

The linked resection instrument 10 includes a femoral pin guide 12 and a tibial pin guide 14 secured to an elongated body 16. The femoral pin guide 12 includes a pair of pin guide holes 22 that are configured to locate the installation of a pair of femoral alignment pins 122 on the anterior surface 124 of the patient’s femur 120 (see FIG. 8). As shown in FIG. 8, a distal femoral cutting block 112 is installed on the femoral alignment pins 122 and thereafter used in the performance of a distal femoral resection on the patient’s femur 120. Similarly, the tibial pin guide 14 includes a pair of pin guide holes 24 that are configured to locate the installation of a pair of tibial alignment pins 132 on the anterior surface 134 of the patient’s tibia 130 (see FIG. 8). As shown in FIG. 8, a tibial cutting block 114 is installed on the tibial alignment pins 132 and thereafter used in the performance of a tibial resection on the patient’s tibia 130.

The femoral pin guide 12 includes a collar 30 with a pair of arms 32 extending mediolaterally outwardly therefrom. One of the pin guide holes 22 is formed near the outer end of each of the arms 32. The collar 30 of the femoral pin guide 12 has an elongated bore 34 extending therethrough. A segment 40 of the elongated body 16 defining the body’s superior end has a smooth outer surface 42 that is sized and shaped to be received into the collar’s bore 34. Thus, the collar 30 – and hence the femoral pin guide 12 – is free to superinferiorly translate along the smooth outer surface 42 of the elongated body 16. As can be seen in FIGS. 2-4, the medial and lateral sides of the superior segment 40 have elongated slots 44 formed therein. A retaining pin 46 passes through the collar 30 and extends through both slots 44 of the elongated body 16 thereby capturing the femoral pin guide 12 on the elongated body 16. The length of the slots 44 also functions to limit the distance of travel of the collar 30.

As can be seen in FIGS. 1-4, a locking knob 50 is used to lock the position of the femoral pin guide 12 relative to the elongated body 16 and hence the tibial pin guide 14. The locking knob 50 has an internal shaft that extends inwardly through the collar 30 and into the bore 34 and is displaced by rotation of the knob 50 into and out of contact with the body’s outer surface 42 to selectively clamp the collar 30 – and hence the femoral pin guide 12 - in a locked position relative to the elongated body 16. In particular, when the locking knob 50 is loosened, the femoral pin guide 12 is free to slide relative to the elongated body 16 so as to adjust the distance between the femoral pin guide 12 and the tibial pin guide 14. Once a desired distance is achieved, the locking knob 50 may be tightened to lock the position of the two pin guides 12, 14 relative to one another. It should be appreciated that such changing of the distance between the two pin guides 12, 14 changes the gap width created by the cutting blocks 112, 114 positioned on the alignment pins 122, 132 installed by use of the pin guides 12, 14. In particular, as seen in FIG. 8, the distal femoral cutting block 112 has a cutting slot 116 that guides the blade of a bone saw (not shown) in making a distal femoral resection. Similarly, the tibial cutting block 114 has a cutting slot 118 that guides the blade in making a tibial resection. The distance between the guide surfaces of the two cutting slots 116, 118 defines the width (GW) of the resultant gap between the two resected bone surfaces. This “gap width,” as it is commonly referred, is predetermined by the surgeon based on the thickness of the knee prosthesis (e.g., the combined thickness of the tibial tray and bearing insert) that the surgeon has selected for implantation. For example, if the surgeon desires to implant a knee prosthesis having a thickness of 10 mm, the surgeon positions the cutting blocks 112, 114 relative to one another such that their cutting slots 116, 118 create a 10 mm gap width. Since the position of the cutting blocks 112, 114 relative to one another is based on the position of the installed femoral alignment pins 122 and tibial alignment pins 132 relative to one another, which are, in turn, based on the position of the pin guides 12, 14 relative to one another, changing the distance between the pin guides 12, 14 changes the gap width (GW) created by the femoral and tibial resections.

As can be seen in FIGS. 1-4, the linked resection instrument 10 includes a slope adjustment collet 52. As will be discussed below in greater detail, the slope adjustment collet 52 may be used by the surgeon to adjust the angle of the tibial pin guide 14 in the anteroposterior direction so as to alter the posterior slope angle of the knee prosthesis implanted on the patient’s tibia 130. The slope adjustment collet 52 has a knurled knob 54 configured to be gripped by the surgeon to rotate the collet 52 relative to the elongated body 16. A cylindrically-shaped display column 56 extends superiorly away from the upper surface of the knob 54. The display column 56 has indicia in the form of a plurality of gap width indicators 58 engraved, printed, or otherwise formed thereon. Each of the plurality of gap width indicators 58 represents the gap width (e.g., 5 mm to 16 mm) that would be created based on a given position of the pin guides 12, 14 relative to one another. The collar 30 of the femoral pin guide 12 has a sleeve 36 extending inferiorly away from the collar’s bottom surface. The outer surface of the sleeve 36 has an indicator line 38 engraved, printed, or otherwise formed thereon. The indicator line 38 is visible to the surgeon via a slot 60 formed in the collet’s display column 56. As either or both of the femoral pin guide 12 and the collet 52 are moved upwardly or downwardly relative to one another, the indicator line 38 moves relative to the collet’s display column 56 so as to align with the gap width indicator 58 that represents the gap width (e.g., 5 mm to 16 mm) that would be created based on the current position of the pin guides 12, 14 relative to one another.

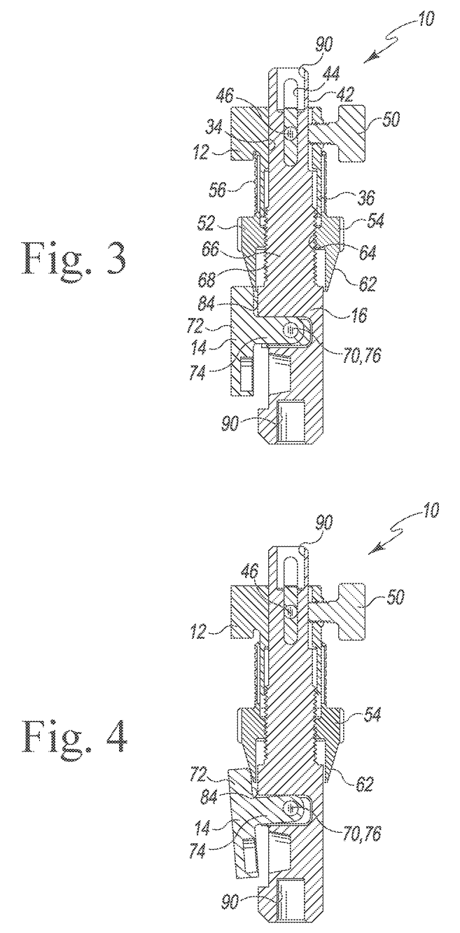

The slope adjustment collet 52 has a tapered outer surface 62 extending inferiorly away from the lower surface of the knob 54. The tapered outer surface 62 tapers downwardly away from the knob 54. In other words, the superior end of the tapered outer surface 62 is wider than its inferior end. As can be seen in FIGS. 3 and 4, the knob 54 of the slope adjustment collet 52 has a threaded bore 64 extending therethrough. A segment 66 of the elongated body 16 defining the body’s middle has a threaded outer surface 68 that is sized and shaped (e.g., pitched) to be threadingly-engaged by the collet’s threaded bore 64. As such, rotation of the knob 54 causes the collet 52 to be translated upwardly or downwardly along the threaded outer surface 68 of the elongated body 16 (based on the direction of rotation).

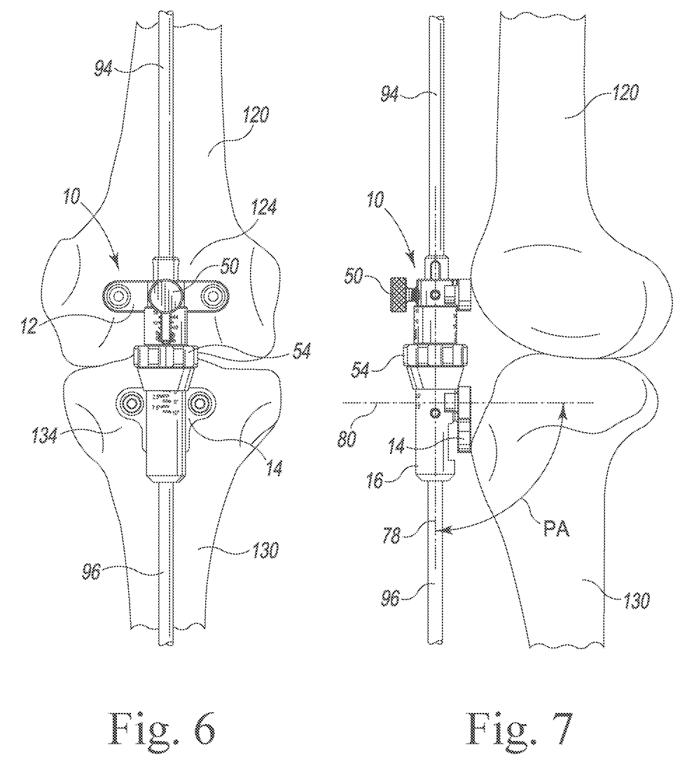

As can be seen in FIGS. 2-4, the tibial pin guide 14 is pivotally coupled to the elongated body 16 at a location inferior to the femoral pin guide 12. The tibial pin guide 14 includes a flange 72 having an arm 74 that is pivotally secured to the elongated body 16 via a pivot joint 70 created by a pivot pin 76. As can be seen in FIG. 1, one of the pin guide holes 24 is formed near each of the outer medial and lateral ends of the flange 72. As shown in FIG. 7, the elongated body 16 has a longitudinal axis 78 that defines an anteroposterior pin angle (PA) with a longitudinal axis 80 of the pin guide holes 24. Rotation of the arm 74 - and hence the flange 72 - relative to the elongated body 16 at the pivot joint 70 selectively alters the anteroposterior pin angle (PA). Altering the anteroposterior pin angle (PA) alters the posterior slope angle of the knee prosthesis to be implanted on the patient’s tibia 130. In particular, as shown in FIG. 7, altering the anteroposterior pin angle (PA) alters the anteroposterior angle in which the tibial alignment pins 132 are installed in the patient’s tibia 130. Altering the anteroposterior angle in which the tibial alignment pins 132 are installed in the patient’s tibia 130 likewise alters the anteroposterior angle of the cutting plane 136 of the cutting slot 118 of the tibial cutting block 114 when the tibial cutting block 114 is installed on the tibial alignment pins 132. By altering the anteroposterior angle of the cutting plane 136 of the cutting slot 118 of the tibial cutting block 114, the anteroposterior angle of the resection made by guiding a bone saw blade (not shown) with the cutting slot 118 is likewise altered. Such an angled resection - commonly referred to as the “posterior slope angle” - determines the anteroposterior angle in which the knee prosthesis is installed on the patient’s tibia 130. The surgeon may select a given posterior slope angle (e.g., 0 degrees to 10 degrees) based on, for example, the type of tibial implant being implanted (e.g., 3 degrees if a posterior stabilized knee prosthetic is being implanted or 5-7 degrees if a cruciate retaining prosthetic is being implanted).

The slope adjustment collet 52 may be used to alter the anteroposterior pin angle (PA) of the tibial pin guide 14 and thus alter the posterior slope angle of the knee prosthesis to be implanted on the patient’s tibia 130. Specifically, rotation of the collet’s knob 54 in one direction (e.g., clockwise) moves the collet 52 downwardly (i.e., inferiorly) along the threaded outer surface 68 of the elongated body 16 so as to urge the collet’s tapered outer surface 62 toward the flange 72 of the tibial pin guide 14. During such downward movement of the collet 52, the tapered outer surface 62 engages a cam surface 84 formed in the anterior face of the superior end of the flange 72. As the tapered outer surface 62 of the collet 52 moves downwardly along the cam surface 84 of the pin guide’s flange 72, the flange 72 pivots about the pivot joint 70 so as to increase the anteroposterior pin angle (PA) of the tibial pin guide 14 (and thus increase the posterior slope angle of the knee prosthesis to be implanted on the patient’s tibia 130). Rotation of the collet’s knob 54 in the opposite direction (e.g., counterclockwise) moves the collet 52 upwardly (i.e., superiorly) along the threaded outer surface 68 of the elongated body 16 so as to urge the collet’s tapered outer surface 62 in the direction away from the flange 72 of the tibial pin guide 14. As the tapered outer surface 62 of the collet 52 moves upwardly along the cam surface 84 of the pin guide’s flange 72, the flange 72 pivots about the pivot joint 70 so as to decrease the anteroposterior pin angle (PA) of the tibial pin guide 14 (and thus decrease the posterior slope angle of the knee prosthesis to be implanted on the patient’s tibia 130).

As can be seen in FIGS. 1 and 2, the elongated body 16 has indicia in the form of a plurality of angle indicators 88 engraved, printed, or otherwise formed thereon. Each of the plurality of angle indicators 88 represents a desired posterior slope angle (e.g., 0 degrees to 10 degrees) for selection by a surgeon during use of the linked resection instrument 10. The inferior-most edge 86 of the collet 52 functions as an indicator, as shown in FIG. 1. Specifically, as the collet 52 is moved upwardly or downwardly along the elongated body 16, the inferior-most edge 86 of the collet 52 aligns with the angle indicator 88 that represents the posterior slope angle (e.g., 0 degrees to 10 degrees) that would be created based on the current anteroposterior pin angle (PA) of the tibial pin guide 14.

As shown in FIGS. 3 and 4, both the superior end and the inferior end of the elongated body 16 have a socket 90 formed therein such that a femoral alignment rod 94 and a tibial alignment rod 96 may be secured to the elongated body 16 and thereafter aligned with the patient’s hip 154 and ankle 156, respectively. In particular, the tibial alignment rod 96 is secured to the socket 90 on the inferior end of the elongated body 16 and extends inferiorly away therefrom toward the patient’s ankle 156. The tibial alignment rod 96 may be used to position the linked resection instrument 10 relative to the patient’s ankle 156 so that the alignment of the patient’s femur 120 and tibia 130 may be assessed. Typically, the instrument 10 is positioned such that the tibial alignment rod 96 extends along the mechanical axis of the patient’s tibia 130 such that it is aligned with the patient’s ankle 156. The femoral alignment rod 94 is secured to the socket 90 in the superior end of the elongated body 16 and extends superiorly away therefrom toward the patient’s hip 154. The femoral alignment rod 94 may be used to position the linked resection instrument 10 relative to the patient’s hip 154 so that the alignment of the patient’s femur 120 and tibia 130 may be further assessed. Typically, the femoral alignment rod 94 is positioned along an axis that is aligned with the center of the patient’s hip 154 so as to allow the surgeon to assess the overall hip-knee-ankle angle when the tibial alignment rod 96 is aligned with the patient’s ankle 156.

The linked resection instrument 10 may be used both by surgeons with a preference for mechanical alignment techniques and those surgeons with a preference for kinematic alignment techniques. Broadly, mechanical alignment techniques involve determining the resection planes based on a pre-determined angle as a function of mechanical alignment, whereas kinematic alignment techniques involve determining the resection planes as a function of the native, pre-disease state of the patient’s knee. In a further example, the linked resection instrument 10 may also be used in an inverse kinematic alignment technique in which ligament balancing is used in conjunction with kinematic alignment techniques. In any such case, the surgeon may use the linked resection instrument 10 to locate the distal femoral cutting block 112 in relation to the location of the tibial cutting block 114, and vice versa. The linked resection instrument 10 may be used by the surgeon to intraoperatively confirm the location of both blocks 112, 114 prior to the performance of a resection with either of them.

In operation, the surgeon may utilize the linked resection instrument 10 during performance of an orthopaedic knee procedure to prepare the distal end of the patient’s femur 120 and the proximal end of the patient’s tibia 130 to receive prosthetic femoral and tibial components. In doing so, the surgeon may utilize the linked resection instrument 10 to install the distal femoral cutting block 112 on the patient’s femur 120 as a function of, or based on, the location and orientation of the tibial cutting block 114 installed on the patient’s tibia 130. Alternatively, the surgeon may utilize the linked resection instrument 10 to install the tibial cutting block 114 on the patient’s tibia 130 as a function of, or based on, the location and orientation of the distal femoral cutting block 112 installed on the patient’s femur 120.

As noted above, in one surgical method the surgeon may determine the location and orientation of the femoral cutting block 112 on the patient’s femur 120 as a function of, or based on, the location and orientation of the tibial cutting block 114 installed on the patient’s tibia 130. To do so, the surgeon first determines the desired location and orientation of the tibial cutting block 114. The surgeon may use any number of techniques – including mechanical alignment techniques and/or kinematic alignment techniques – to locate the tibial cutting block 114 on the patient’s tibia 130. In one example, the surgeon may use an alignment instrument such as the Attune® Tibial Alignment Jig which is commercially available from DePuy Synthes of Warsaw, Indiana. In another example, the surgeon may use the tibial alignment jig instrument and techniques taught in co-pending U.S. Patent Appl. Ser. No. 63/741,632 entitled “TIBIAL ALIGNMENT JIG INSTRUMENT AND METHOD OF USING THE SAME IN AN ORTHOPAEDIC SURGICAL KNEE PROCEDURE” which was filed on Jan. 3, 2025, is assigned to the same assignee as the present disclosure, and is hereby incorporated herein in its entirety. In such techniques, the tibial cutting block 114 is aligned by use of the instrument and then pinned in place using the pair of tibial alignment pins 132.

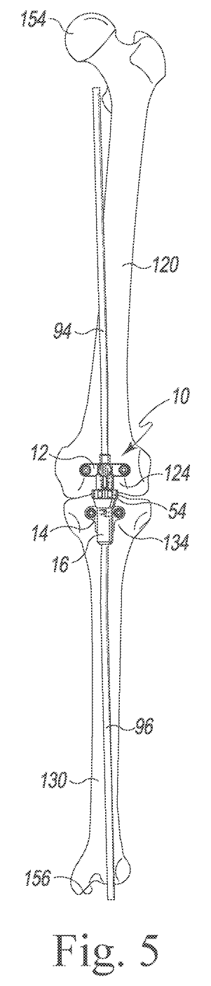

After the surgeon has located and pinned the tibial cutting block 114 in place on the anterior surface 134 of the patient’s tibia 130, the surgeon then removes the tibial cutting block 114 from the installed tibial alignment pins 132. Thereafter, if not already so positioned, the surgeon orientates the patient’s femur 120 and tibia 130 such that the patient’s knee is positioned in extension, as shown in FIGS. 5-7. With the patient’s knee positioned in extension, the surgeon installs the linked resection instrument 10 on the previously installed tibial alignment pins 132. Specifically, the surgeon aligns the pin guide holes 24 of the linked resection instrument’s tibial pin guide 14 with the tibial alignment pins 132 installed in the patient’s tibia 130 and thereafter advances the linked resection instrument 10 such that the pins 132 are received into the pin guide holes 24. The surgeon advances the instrument 10 until the posterior side of the tibial pin guide 14 abuts the anterior surface 134 of the patient’s tibia 130. Doing so also causes the posterior side of the femoral pin guide 12 to abut the anterior surface 124 of the patient’s femur 120.

If not already done prior to installation of the linked resection instrument 10, the surgeon configures the instrument 10 with initial settings based on the size and type of knee implant to be installed. Specifically, the surgeon loosens the locking knob 50 and adjusts the distance between the femoral pin guide 12 and the tibial pin guide 14 to create a desired gap width (GW) based on the thickness of the knee prosthesis (e.g., the combined thickness of the tibial tray and bearing insert) that the surgeon has selected for implantation. For example, if the surgeon desires to implant a knee prosthesis having a thickness of 10 mm, the surgeon uses the pin guides 12, 14 to position the cutting blocks 112, 114 relative to one another such that their cutting slots 116, 118 create a 10 mm gap width. As the surgeon adjusts the position of the pin guides 12, 14 relative to one another, the femoral pin guide’s indicator line 38 moves relative to the collet’s display column 56 so as to align with the gap width indicator 58 that represents the gap width (e.g., 5 mm to 16 mm) that would be created based on the current position of the pin guides 12, 14 relative to one another. The surgeon adjusts the position of the pin guides 12, 14 until the indicator line 38 is aligned with the desired gap width indicator 58 and thereafter tightens the locking knob 50 to lock the pin guides 12, 14 in the desired distance from one another (e.g., if the surgeon desires to implant a knee prosthesis having a thickness of 10 mm, the surgeon aligns the indicator line 38 with the 10 mm gap width indicator 58). The surgeon also sets the posterior slope angle to an initial setting based on the type of tibial implant being implanted (e.g., 3 degrees if a posterior stabilized knee prosthetic is being implanted or 5-7 degrees if a cruciate retaining prosthetic is being implanted). To do so, the surgeon rotates the collet’s knob 54 in a direction (e.g., clockwise) that moves the collet 52 downwardly (i.e., inferiorly) along the threaded outer surface 68 of the elongated body 16 so as to urge the collet’s tapered outer surface 62 toward the flange 72 of the tibial pin guide 14. During such downward movement of the collet 52, the tapered outer surface 62 engages the cam surface 84 formed in the anterior face of the superior end of the flange 72. As the tapered outer surface 62 of the collet 52 moves downwardly along the cam surface 84 of the pin guide’s flange 72, the flange 72 pivots about the pivot joint 70 so as to increase the anteroposterior pin angle (PA) of the tibial pin guide 14 (and thus increase the posterior slope angle of the knee prosthesis to be implanted on the patient’s tibia 130). The surgeon continues to rotate the collet’s knob 54 until the inferior-most edge 86 of the collet 52 aligns with the angle indicator 88 that represents the desired posterior slope angle (e.g., 0 degrees to 10 degrees).

With the linked resection instrument 10 set to the surgeon initial settings and installed on the tibial alignment pins 132, the surgeon uses the femoral alignment rod 94 and the tibial alignment rod 96 to assess the overall alignment created by the current position of the linked resection instrument 10. In particular, the tibial alignment rod 96 extends inferiorly along the mechanical axis of the patient’s tibia 130 such that it is aligned with the patient’s ankle 156 and the femoral alignment rod 94 is positioned along an axis that is aligned with the center of the patient’s hip 154 so as to allow the surgeon to assess the overall hip-knee-ankle angle when the tibial alignment rod 96 is aligned with the patient’s ankle 156.

Based on the surgeon’s assessment, the surgeon may opt to adjust one or both of the gap width and the posterior slope angle created by the instrument 10. To alter the gap width, the surgeon may loosen the locking knob 50 and adjust the gap width in the matter described above. Similarly, the surgeon may rotate the collet’s knob 54 to increase or decrease the posterior slope angle. Because the gap width indicators 58 are formed on the collet’s display column 56 – and thus move upwardly and downwardly with the knob 54 – any increase or decrease in the gap width caused by changes to the posterior slope angle are made visible to the surgeon since the gap width indicators 58 move relative to the indicator line 38 as the collet 52 moves relative to the femoral pin guide’s sleeve 36. As such, the surgeon is made aware of any increase or decrease in the gap width caused by adjustment of the posterior slope angle. The surgeon can either accept such a change (and thus install a different size of implant relative to the initial size) or, more commonly, the surgeon can adjust the gap width back to its original intended size by use of the locking knob 50. For example, if the surgeon increases the posterior slope angle and thus causes the gap width to increase to 12 mm from the preoperatively planned 10 mm gap width, the surgeon can loosen the locking knob 50 and move the femoral pin guide 12 back to the 10 mm gap width setting.

Once the surgeon is satisfied with the assessment, the surgeon installs a femoral alignment pin 122 in each of the pin guide holes 22 of the femoral pin guide 12. The surgeon then removes the linked resection instrument 10 leaving the femoral alignment pins 122 in the patient’s femur 120 and the tibial alignment pins 132 in the patient’s tibia 130. Thereafter, as shown in FIG. 8, the surgeon installs the distal femoral cutting block 112 on the femoral alignment pins 122 and the tibial cutting block on the tibial alignment pins 132.

The surgeon may then use the installed tibial cutting block 114 to perform a proximal resection of the proximal end of the patient’s tibia 130 by advancing a bone saw blade (not shown) of a surgical saw through the cutting slot 118 to engage the patient’s tibia 130 and operate the surgical saw to surgically form a planar resected surface of the patient’s tibia 130. Either before or after performing the tibial resection, the surgeon may use the distal femoral cutting block 112 to perform a distal resection of the distal end of the patient’s femur 120. Specifically, the surgeon may advance the bone saw blade of the surgical saw through the distal cutting slot 116 to engage the patient’s femur 120 and operate the surgical saw to surgically form a planar distal resected surface of the patient’s femur 120. The surgeon may then pin an additional cutting block, such as a 4-in-1 cutting block (not shown), on the surgically-prepared distal resected surface and thereafter perform additional resections such as an anterior cut, a posterior cut, and a pair of chamfer cuts. Once the anterior cut, posterior cut, and both chamfer cuts have been made, the surgeon removes the 4-in-1 cutting block from the patient’s femur 120. The surgeon may then perform the remaining surgical steps to complete the orthopaedic knee procedure.

A surgeon with a preference for an inverse kinematic technique may follow a very similar workflow to as described above. The only difference is the process the surgeon uses during the surgeon’s assessment after the linked resection instrument 10 is installed on the tibial alignment pins 132. In the case of an inverse kinematic technique, the surgeon installs a ligament balancer (not shown) between the patient’s femur 120 and tibia 130 and adjusts the alignment of the linked resection instrument 10 while the patient’s knee is balanced via use of the ligament balancer. Once such ligament balancer that may be used in such a technique is described in co-pending U.S. Patent Appl. Ser. No. 63/741,615 entitled “LIGAMENT BALANCED PIN GUIDE INSTRUMENT AND METHOD OF USING THE SAME IN AN ORTHOPAEDIC SURGICAL KNEE PROCEDURE” which was filed on Jan. 3, 2025, is assigned to the same assignee as the present disclosure, and is hereby incorporated herein in its entirety.

As noted above, based on surgeon preferences and/or the needs of a given patient, the surgeon may opt to determine the location and orientation of the tibial cutting block 114 installed on the patient’s tibia 130 as a function of, or based on, the location and orientation of the femoral cutting block 112 installed on the patient’s femur 120. To do so, the surgeon first determines the desired location and orientation of the distal femoral cutting block 112. The surgeon may use any number of techniques – including mechanical alignment techniques and/or kinematic alignment techniques – to locate the distal femoral cutting block 112 on the patient’s femur 120. In one example, the surgeon may use an alignment instrument such as the Attune® Distal Femoral Jig which is commercially available from DePuy Synthes. In another example, the surgeon may use the femoral alignment jig instrument and techniques taught in co-pending U.S. Patent Appl. Ser. No. 63/741,619 entitled “DISTAL FEMORAL JIG INSTRUMENT AND METHOD OF USING THE SAME IN AN ORTHOPAEDIC SURGICAL KNEE PROCEDURE” which was filed on Jan. 3, 2025, is assigned to the same assignee as the present disclosure, and is hereby incorporated herein in its entirety. In such techniques, the distal femoral cutting block 112 is aligned by use of the instrument and then pinned in place using the pair of femoral alignment pins 122.

After the surgeon has located and pinned the femoral cutting block 112 in place on the anterior surface 124 of the patient’s femur 120, the surgeon then removes the distal femoral cutting block 112 from the installed femoral alignment pins 122. Thereafter, if not already so positioned, the surgeon orientates the patient’s femur 120 and tibia 130 such that the patient’s knee is positioned in extension, as shown in FIGS. 5-7. With the patient’s knee positioned in extension, the surgeon installs the linked resection instrument 10 on the previously installed femoral alignment pins 122. Specifically, the surgeon aligns the pin guide holes 22 of the linked resection instrument’s femoral pin guide 12 with the femoral alignment pins 122 installed in the patient’s femur 120 and thereafter advances the linked resection instrument 10 such that the pins 122 are received into the pin guide holes 22. The surgeon advances the instrument 10 until the posterior side of the femoral pin guide 12 abuts the anterior surface 124 of the patient’s femur 120. Doing so also causes the posterior side of the tibial pin guide 14 to abut the anterior surface 134 of the patient’s tibia 130.

If not already done prior to installation of the linked resection instrument 10, the surgeon configures the instrument 10 with initial settings based on the size and type of knee implant to be installed, in the manner described above. The surgeon then uses the femoral alignment rod 94 and the tibial alignment rod 96 to assess the overall alignment created by the current position of the linked resection instrument 10, in the manner described above. If need be, the surgeon may adjust the gap width and posterior slope angle in a similar manner to as described above. Once the surgeon is satisfied with the assessment, the surgeon installs a tibial alignment pin 132 in each of the pin guide holes 24 of the tibial pin guide 14. The surgeon then removes the linked resection instrument 10, installs the distal femoral cutting block 112 and the tibial cutting block 114, and performs both resections in a similar manner to as described above.

In the techniques described above, either the distal femoral cutting block 112 or the tibial cutting block 114 is first placed on the patient’s femur 120 or tibia 130 by use of, for example, an alignment jig with the other block 112, 114 then being placed by use of the linked resection instrument 10 as a function of, or based on, the location and orientation of the previously installed block 112, 114. In a further example, the linked resection instrument 10 alone may be used to place both blocks 112, 114. To do so, the surgeon first orientates the patient’s femur 120 and tibia 130 such that the patient’s knee is positioned in extension, as shown in FIGS. 5-7. With the patient’s knee positioned in extension, the surgeon positions the linked resection instrument 10 on the patient’s femur 120 and tibia 130 and uses the femoral alignment rod 94 and the tibial alignment rod 96 to set the initial position of the linked resection instrument 10.

If not already done prior to installation of the linked resection instrument 10, the surgeon configures the instrument 10 with initial settings based on the size and type of knee implant to be installed. The surgeon then uses the femoral alignment rod 94 and the tibial alignment rod 96 to assess the overall alignment created by the current position of the linked resection instrument 10 in the manner described above. If need be, the surgeon may adjust the gap width and posterior slope angle in a similar manner to as described above. Once the surgeon is satisfied with the assessment, the surgeon installs a tibial alignment pin 132 in each of the pin guide holes 24 of the tibial pin guide 14 and a femoral alignment pin 122 in each of the pin guide holes 22 of the femoral pin guide 12. The surgeon then removes the linked resection instrument 10, installs the distal femoral cutting block 112 and the tibial cutting block 114, and performs both resections in a similar manner to as described above.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. An orthopaedic surgical instrument assembly for use during an orthopaedic knee replacement procedure, comprising:
an elongated body,
a femoral pin guide positioned on the elongated body, the femoral pin guide being slidable along the elongated body in the superoinferior direction,
a tibial pin guide pivotally coupled to the elongated body at a location inferior to the femoral pin guide, and
a slope adjustment collet rotatably coupled to the elongated body, wherein (i) the slope adjustment collet has a tapered outer surface, (ii) rotation of the slope adjustment collet in a first direction urges the collet’s tapered outer surface toward the tibial pin guide so as to increase an anteroposterior pin angle of the tibial pin guide, and (iii) rotation of the slope adjustment collet in a second, opposite direction urges the collet’s tapered outer surface away from the tibial pin guide so as to decrease the anteroposterior pin angle of the tibial pin guide.

2. The orthopaedic surgical instrument assembly of claim 1, wherein the femoral pin guide comprises a pair of pin guide holes configured to locate placement of a distal femoral cutting block on an anterior surface of a femur of a patient.

3. The orthopaedic surgical instrument assembly of claim 1, wherein:
the tibial pin guide comprises a flange having a pair of pin guide holes configured to locate placement of a tibial cutting block on an anterior surface of a tibia of a patient,
the elongated body has a longitudinal axis that defines the anteroposterior pin angle with a longitudinal axis of the pair of pin guide holes, and
the flange is pivotally coupled to the elongated body via a pivot joint,
rotation of the flange relative to the elongated body at the pivot joint selectively alters the anteroposterior pin angle.

4. The orthopaedic surgical instrument assembly of claim 1, wherein:
the tibial pin guide has a cam surface formed therein,
rotation of the slope adjustment collet in the first direction moves the collet’s tapered outer surface inferiorly along the cam surface of the tibial pin guide so as to increase the anteroposterior pin angle of the tibial pin guide, and
rotation of the slope adjustment collet in the second, opposite direction urges the collet’s tapered outer surface superiorly along the cam surface of the tibial pin guide so as to decrease the anteroposterior pin angle of the tibial pin guide.

5. The orthopaedic surgical instrument assembly of claim 1, wherein:
the elongated body has a superior body segment having a smooth outer surface and a middle body segment having a threaded outer surface,
the femoral pin guide has an elongated bore that is positioned on the superior body segment of the elongated body so as to allow the femoral pin guide to superinferiorly translate along the smooth outer surface, and
the slope adjustment collet has a threaded bore that is threadingly-engaged with the threaded outer surface so as to allow the slope adjustment collet to superinferiorly translate along the elongated body’s middle body segment.

6. The orthopaedic surgical instrument assembly of claim 1, further comprising a locking knob operable to lock the position of the femoral pin guide relative to the elongated body.

7. The orthopaedic surgical instrument assembly of claim 1, further comprising:
a femoral alignment rod secured to a superior end of the elongated body and extending superiorly away therefrom, and
a tibial alignment rod secured to an inferior end of the elongated body and extending inferiorly away therefrom.

8. A method of surgically preparing a patient’s femur and tibia during an orthopaedic surgical knee procedure, comprising:
installing a pair of femoral alignment pins in an anterior surface of the patient’s femur,
installing a femoral pin guide of a linked resection instrument onto the pair of installed femoral alignment pins,
adjusting a distance between the femoral pin guide of the linked resection instrument and a tibial pin guide of the linked resection instrument based on a thickness of a knee prosthesis to be implanted,
adjusting an anteroposterior pin angle of the tibial pin guide of the linked resection instrument based on a desired posterior slope angle of the knee prosthesis to be implanted, and
installing a pair of tibial alignment pins in an anterior surface of the patient’s tibia with the tibial pin guide of the linked resection instrument.

9. The method of claim 8, wherein installing the pair of femoral alignment pins comprises operating a femoral alignment jig to install the pair of femoral alignment pins in the anterior surface of the patient’s femur.

10. The method of claim 8, further comprising:
aligning a tibial alignment rod of the linked resection instrument with an ankle of the patient, and
aligning a femoral alignment rod of the linked resection instrument with a hip of the patient.

11. The method of claim 8, further comprising:
assessing changes to the distance between the femoral pin guide of the linked resection instrument and the tibial pin guide of the linked resection instrument as a result of adjustment of the anteroposterior pin angle, and
readjusting the distance between the femoral pin guide of the linked resection instrument and the tibial pin guide of the linked resection instrument.

12. The method of claim 8, further comprising:
installing a distal femoral cutting block on the installed femoral alignment pins, and
resecting a distal end of the patient’s femur with the installed distal femoral cutting block.

13. The method of claim 8, further comprising:
installing a tibial cutting block on the installed tibial alignment pins, and
resecting a proximal end of the patient’s tibia with the installed tibial cutting block.

14. A method of surgically preparing a patient’s femur and tibia during an orthopaedic surgical knee procedure, comprising:
installing a pair of tibial alignment pins in an anterior surface of the patient’s tibia,
installing a tibial pin guide of a linked resection instrument onto the pair of installed tibial alignment pins,
adjusting a distance between the tibial pin guide of the linked resection instrument and a femoral pin guide of the linked resection instrument based on a thickness of a knee prosthesis to be implanted,
adjusting an anteroposterior pin angle of the tibial pin guide of the linked resection instrument based on a desired posterior slope angle of the knee prosthesis to be implanted, and
installing a pair of femoral alignment pins in an anterior surface of the patient’s femur with the femoral pin guide of the linked resection instrument.

15. The method of claim 14, wherein installing the pair of tibial alignment pins comprises operating a tibial alignment jig to install the pair of tibial alignment pins in the anterior surface of the patient’s tibia.

16. The method of claim 14, further comprising:
aligning a tibial alignment rod of the linked resection instrument with an ankle of the patient, and
aligning a femoral alignment rod of the linked resection instrument with a hip of the patient.

17. The method of claim 14, further comprising:
assessing changes to the distance between the tibial pin guide of the linked resection instrument and the femoral pin guide of the linked resection instrument as a result of adjustment of the anteroposterior pin angle, and
readjusting the distance between the tibial pin guide of the linked resection instrument and the femoral pin guide of the linked resection instrument.

18. The method of claim 14, further comprising:
installing a distal femoral cutting block on the installed femoral alignment pins, and
resecting a distal end of the patient’s femur with the installed distal femoral cutting block.

19. The method of claim 14, further comprising:
installing a tibial cutting block on the installed tibial alignment pins, and
resecting a proximal end of the patient’s tibia with the installed tibial cutting block.

20. The method of claim 14, further comprising:
installing a ligament balancer between the patient’s tibia and femur before installing the pair of femoral alignment pins in the anterior surface of the patient’s femur with the femoral pin guide of the linked resection instrument.
